(19) United States
(12) Patent Application Publication
(10) Pub. No.: US 2007/0211847 A1
(43) Pub. Date: Sep. 13, 2007

(54) METHOD FOR RECORDING PROJECTION DATA SETS OF AN OBJECT UNDER EXAMINATION

(76) Inventors: Rainer Graumann, Hochstadt (DE); Erwin Lutz, Poxdorf (DE)

Correspondence Address: SCHIFF HARDIN, LLP;PATENT DEPARTMENT, 6600 SEARS TOWER, CHICAGO, IL, 60606-6473, US

(21) Appl. No.: 11/684,077

(22) Filed: Mar. 9, 2007

(30) Foreign Application Priority Data
Mar. 10, 2006 (DE) 10 2006 011 235.0

(51) Int. Cl. A61B 6/00; G01N 23/00; G21K 1/12; H05G 1/60

(52) U.S. Cl. 378/15

(57) ABSTRACT

In a method for recording radiological projection data sets of an object under examination, a number of two-dimensional projection data sets of the object under examination being recorded, which are characterized by an axis of rotation having a spatial position, with a projection data set being obtained from an x-ray beam penetrating the object under examination, which essentially diffuses at a right angle to the axis of rotation. By changing the spatial position of the axis of rotation between the recording of two successive projection data sets, increases the versatility of the x-ray device increased, in particular for a movable x-ray device, and the image quality of the spatial representations that are reconstructed from the projection data sets is improved.

### BACKGROUND OF THE INVENTION

1. Field of the Invention

The present invention relates to a method for recording radiological projection data sets of an object under examination, with a number of two-dimensional projection data sets of the object under examination being recorded, which are characterized by an axis of rotation having a spatial position, with a projection data set being obtained from an x-ray beam penetrating the object under examination, which essentially diffuses at a right angle to the axis of rotation.

2. Description of the Prior Art

Despite advancing developments and new possibilities in the field of radiation-free medical diagnostics, x-ray devices still represent an important source of support in medical technology. X-ray devices can be found in a large number of medical applications ranging from x-ray diagnostics, for example for clarification of bone fractures, tumors, cysts, calcifications, or trapped air, as well as precautionary examinations, fluoroscopic examinations, for example in angiography, as well as monitoring medical interventions or localization of medical instruments, etc. X-ray devices of the type initially described are frequently C-arm x-ray devices, which are becoming increasingly established in medical applications due to the advantages they offer.

The advantages of C-arm x-ray devices include the possibility of spatial representations of an object or a patient with simultaneous good access to the patient, which is of particular importance in medical interventions. The possibility further exists to realize C-arm x-ray devices in a mobile embodiment, thus increasing their versatility—for use with bedridden patients for example—and thus reducing costs and overhead. In order to obtain a spatial representation of a patient, such a section of the patient or an organ therein, the C-arm is generally rotated through its range around the patient by means of a motorized drive. During the rotation a series of two-dimensional, isocentric projections of the object under examination is recorded using x-ray radiation from various projection directions, with the angular range of overlap by the orbital movement equaling approximately 200° or more. The term “orbital movement” is used herein according to its conventional meaning of rotation around an axis that intersects the plane of the C-arm at right angles. The recording of a number of projections, or of the projection data sets assigned to the projections to obtain a spatial representation, is also known as an examination sequence. Following the examination sequence, a spatial representation of the body part being examined is obtained from the recorded projection data sets by means of a reconstruction method.

X-ray recording devices equipped with a C-arm also exist that obtain a spatial representation of a section of a patient by rotating the x-ray emitter and the x-ray detector around an angulation axis in the plane of the C-arm and perpendicular to the orbital axis. Such x-ray devices have the disadvantage that only peripheral areas of the patients body, or the patient's extremities, can be examined.

When using x-ray devices to obtain a spatial representation of an object under examination, circumstances may arise that impede, restrict or rule out the possibility of obtaining spatial representations using known methods. For example, metal struts that form part of the patient positioning device may be present, which can absorb the x-ray radiation in certain projection directions corresponding to the directions of the respective x-ray beam, and thus can become undesirably included in the recorded projection data sets. A fault-free reconstruction of spatial representations of the object under examination is thus no longer possible with such projection data sets. A spatial limitation of the rotatability of the recording device, for example a C-arm with x-ray emitter and x-ray detector, is also conceivable during orbital rotation or angular rotation, with the effect that a complete data set of projection data sets for reconstruction of the spatial representation using known methods cannot be provided for the C-arm's initial position. Examples of such limitations include walls, stationary medical devices, and so on. While the x-ray devices currently in use feature a number of degrees of rotational freedom, during the examination sequence they are committed to only one axis of rotation around which the recording device being used to record the projection data sets, generally in the form of an x-ray emitter and an x-ray detector, is rotated.

German application DE 197 46 092 A1 discloses an x-ray recording device having an x-ray source and an x-ray receiver, which are movable in order to record successive 2D projections of an object from various projection directions relative to the object, and having means to generate a 3D image data set from the recorded 2D projections. This disclosed x-ray recording device has the disadvantage of reduced versatility, since the 2D projections recorded during the examination during one rotation are recorded around one common axis of rotation for all 2D projections. It is thus not possible to respond flexibly to the circumstances of the examination.

### SUMMARY OF THE INVENTION

An object of the present invention is to provide a method of the type initially described that allows versatility of the x-ray device, in particular a movable x-ray device, to be increased. A further object of the invention is to improve the image quality for spatial representations of an object under examination with such a method.

This object is achieved in accordance with the invention by a method of the type initially described, but wherein the spatial position of the axis of rotation between the recording of two successive projection data sets is changed. This enables a number of positions of the axis of rotation to be realized and thus allows the possibility of recording projection data sets to be significantly increased. For example, it is no possible to overlay several rotation movements and possibly translation movements in order to obtain a spatial representation of an object under examination. The axis of rotation is thus freely movable in its position. However, all the axes of rotation used during the examination sequence generally run through a common point: the isocenter of the examination. A further advantage of the method is that it can be transferred to known x-ray devices for example in the form of a control program. In particular the inventive method can be advantageously used for C-arm x-ray devices. The versatility of known x-ray devices can thus be significantly and cost-effectively increased, whereby the quality of the spatial representations that can be obtained under the given circumstances can also be significantly improved. Versatility in this context is taken to mean a method-related and device-related multiplicity of examination options that achieve the goal of the examination under variable examination conditions or examination circumstances.

In an embodiment of the invention a second rotary motion around a second essentially horizontal axis of rotation at a right angle to the first axis of rotation is overlaid on to a first rotary motion around a first essentially horizontal axis of rotation. By overlaying such axes of rotation, which are available in particular in the case of C-arm x-ray devices as the angulation axis in the plane of the C-arm and the orbital axis at a right angle to the plane of the C-arm, the inventive method can be implemented with little overhead. By overlaying the rotary motion around the angulation axis and around the orbital axis when recording projection data sets, a number of previously immovable projection geometries can be achieved during a single examination sequence. It is thus also possible to respond more flexibly to circumstances such as interfering objects that can obstruct the recording of projection data sets in the familiar manner—i.e. rotation around either an angulation axis or an orbital axis—and it is also possible to take such circumstances into account in the examination sequence.

In a preferred embodiment of the invention the change in the position of the axis of rotation is automatically controlled. In order to obtain spatial representations of an object under examination it is generally necessary to know the projection geometries when recording projection data sets, since they are incorporated in the reconstruction method as calculation parameters. With the inventive method in particular it is advantageous to perform this potentially complicated movement in a controlled manner on account of the potentially complicated movements in the path of the x-ray emitter and the x-ray detector which are possible as a result of changing the axis of rotation during the examination sequence. The control device allows for precise manipulation of the recording device, with the projection geometries for the respective recorded projection data set being stored in a control device for controlling the movement and subsequently being able to be put to use in order to implement the reconstruction method.

In a further embodiment of the invention the change in the position of the axis of rotation is performed manually. Thus a medical person skilled in the art is given the possibility of performing manual recordings with specific projection geometry. This may be necessary for example in order to test the recording device's rotational freedom or the obstruction of same with regard to the general conditions without incurring the risk of a collision. The recording device can also be manually positioned in a specially desired manner for example for individual recordings.

In a preferred embodiment of the invention the change in the position of the axis of rotation is performed in a motorized manner. Through a motorized change in the position of the axis of rotation, the two-dimensional projection data sets required in order to obtain the spatial representation can be recorded with little time overhead. The motorized change in the position of the axis of rotation allows for a movement of the recording device that is defined and controlled by a control device, which among other things increases the plane of safety both for the x-ray device, for example with regard to slippage of the recording device during a manual change in the position of the axis of rotation, and for the object under examination.

In a further embodiment of the invention planning for the change in the position of the axis of rotation takes place prior to the change in the position of the axis of rotation. In view of the prevailing general conditions, such as metal struts in a patient positioning device, or interfering objects, and of the complicated movement of the recording device, which includes, for example, an overlaid rotation around two axes of rotation positioned at right angles to one another, planning the examination sequence reduces erroneous results, for example in the event that metal struts are included in the recording as a result of the choice of projection geometry when recording the projection data sets, or that a collision takes place with an interfering object. Planning the examination sequence thus reduces the risk of endangering the x-ray device, other objects and people. Planning can advantageously be performed by medical personnel on an input/output unit located on the x-ray device, taking account of the purpose of the examination and the prevailing environmental circumstances.

In a further advantageous embodiment of the invention the change in the position of the axis of rotation is restricted to the change in the position of the axis of rotation identified during planning. The results of the planning are fed to the control device of the x-ray device and/or the recording device and stored. In the course of planning, spatial regions for example are defined within which the recording device can move, for example from the point of view of the person performing the planning, without a collision occurring. The control device, which has access to the planning results, controls a manual or motorized change in the position of the axis of rotation such that the recording device can only be moved within the defined spatial region. Thus it is possible to ensure that the planning results are not infringed and that a collision does not occur as a consequence.

In a further embodiment of the invention a computerized simulation of the change in the position of the axis of rotation takes place prior to the change in the position of the axis of rotation. The simulation can be performed on the basis of the planning results but can also be performed independently, for example because planning is not considered by the medical personnel to be necessary. It is advantageous in this connection to record the circumstances of the examinations, for example using sensors which record the positions of interfering objects within the space and their distances from movable device components of the x-ray device, or also by entry of circumstances into the control device by personnel. A simulation of the examination and/or of the change in the position of the axis of rotation and thus of the change in the position of the recording device is subsequently performed. On the basis of the result of the simulation a decision is made as to whether the examination sequence will be performed in the same manner as the simulation. Performing the simulation increases the plane of safety for x-ray device, objects and people, since an assessment takes place of the examination sequence and/or the planning performed.

In a further embodiment the distance of a first device component to a second device component and/or an interfering object is recorded before or during the change in the spatial position of the axis of rotation. Thus the plane of safety of the x-ray device of the object under examination, and where applicable of interfering objects, is increased further. Interfering objects can include all spatial objects that can compromise the examination sequence of the recording device through their spatial extent, their location and/or their position, which can thus also include the object under examination. They thus include walls, other devices, device components of the x-ray device, and so on. If for example the distance between two device components of the x-ray device, for example the x-ray detector and the stand unit of the x-ray device, falls below a minimum distance the examination sequence is interrupted in a controlled manner and/or the movement of the recording device is halted in order to avoid damage to the device components.

### DESCRIPTION OF THE DRAWINGS

FIG. 1 schematically illustrates an x-ray device for implementing the inventive method.

FIG. 2 shows an angular frequency vector parallelogram for explaining the inventive method.

FIG. 3 is a flowchart of an embodiment of an execution sequence of the inventive method in the form of diagrams.

### DESCRIPTION OF THE PREFERRED EMBODIMENTS

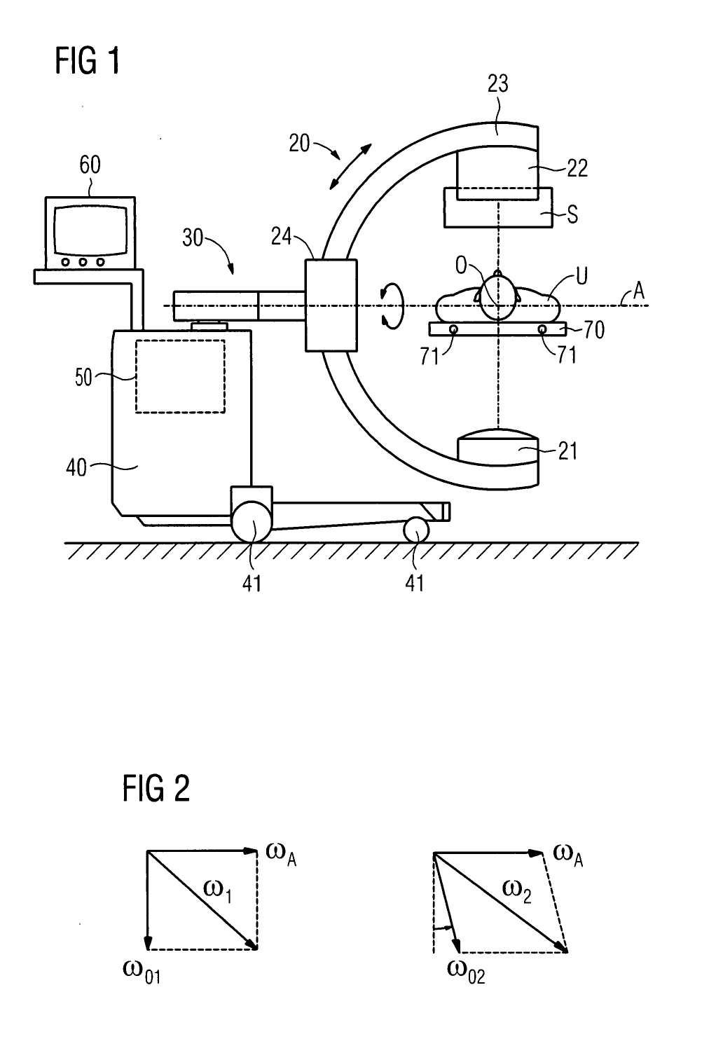

FIG. 1 shows an x-ray device 10 in the form of a movable C-arm x-ray device 10. The C-arm x-ray device 10 has a recording (data acquisition) device 20 with which a number of projection data sets from various projection directions can be recorded. The recording device 20 has an x-ray emitter 21 and an x-ray detector 22, which are arranged opposite to and aligned with one another on a C-arm 23. The C-arm 23 is mounted on a drive device 24. Using the drive device 24 the C-arm 23 can be actuated in a motorized manner along its range around an orbital axis of rotation O positioned at a right angle to the plane of the C-arm, and around a horizontal angulation axis A running in the plane of the C-arm.

The drive device 24 is connected to a stand unit 40 via a holder 30 of any suitable design that allows, for example, translations of the C-arm 23 in the horizontal and vertical direction as well as rotations around one or several vertical axes of rotation. The stand unit 40 is designed so that the x-ray device 10 assumes a stable position even if the recording device 20 moves. Roller elements 41 on which the C-arm x-ray device 10 can be moved are arranged on the underside of the stand unit 40. The stand unit 40 has a control device 50 that is designed to be programmable from a memory. The control device 50 includes a data processor (not shown) and is effectively connected to the drive device 24 and the recording device 20. Recorded projection data sets and the projection geometry associated with the corresponding projection data set are fed to and stored by the control device 50. Once the recording of the projection data sets is complete, a spatial representation of the area under examination is reconstructed from the stored projection data sets in conjunction with the stored associated projection geometries and displayed on the input/output device 60.

In order for an object under examination U to be examined, the object under examination U and/or the recording device 20 and/or the C-arm x-ray device 10 are positioned such that the object under examination U is arranged between x-ray emitter 21 and x-ray detector 22. In order for a spatial representation of an area under examination of the object under examination U to be obtained, it is necessary for a number of projections from various project directions to be recorded. In the event that no obstruction to the rotary motion is expected for example with regard to the rotation around the orbital axis O or the angulation axis A, the number of projection data sets can be recorded in order to obtain the spatial representation according to known methods, in other words rotary motion around the orbital axis O or around the angulation axis A.

In the arrangement of the C-arm x-ray device 10 shown in FIG. 1 the recording of the number of projection data sets by means of rotation around a single axis, either orbital axis O or angulation axis A, is not possible. Two x-ray-absorbent metal struts 71 are present in the patient positioning device 70, which distort the data for the object under examination U. Furthermore an irremovable interfering object S is present that restricts the possibility of rotation around the angulation axis. Under these circumstances an adequate number of undistorted projection data sets cannot be recorded either around the angulation axis A or around the orbital axis O. Consequently a spatial representation of the area under examination cannot be obtained using known methods to a sufficient plane of quality.

The inventive method can be employed, however, which provides for a number of two-dimensional projection data sets with various projection directions to be recorded, in spite of the obstructions shown in FIG. 1, so that a spatial representation of the area under examination to be obtained from the projection data sets. During the examination sequence the position of the axis of rotation is accordingly changed at least once between the recording of a first projection data set and a second projection data set.

FIG. 2 shows the overlaying of the angular frequency vectors of the orbital rotation section ωₒ₁ and ωₒ₂ and of the angular rotation section ω_{A} to an overall angular frequency vectors ω₁ and ω₂ resulting from vector addition during the period of recording a first projection data set and a second projection data set. In the event that the orbital and angular rotations of a C-arm are overlaid, for example, for the C-arm 23 known from FIG. 1, the C-arm continuously or incrementally changes the direction of its axis of rotation. This means that the position of the axis of rotation is not constant during the examination sequence, in contrast to known methods. For example, the axis of rotation is located in position ω1 during recording of the first projection data set. If a second projection data set is now recorded at a corresponding immediately subsequent point in time, the position of the angular frequency vector of the associated orbital rotation section ωₒ₂ has changed—for example rotated—with respect to the position of the angular frequency vector of the orbital rotation section ωo1 associated with the first projection data set, since the angular rotary motion has progressed and the plane of the C-arm, which remains at a right angle to the orbital axis, has rotated further.

Due to the change in the position of the axis of rotation, the x-ray emitter and x-ray detector arranged on a C-arm has the possibility of moving virtually freely during the examination sequence on a spherical surface around an object under examination and accordingly possesses a number of degrees of freedom for recording projection data sets, which are suitable in order to obtain spatial representations. In contrast to hitherto known methods the movement of an x-ray emitter and an x-ray detector during an examination sequence is not restricted just to selected equatorial circumferences, the equatorial circular areas of which are arranged at a right angle to the corresponding axis of rotation. Consequently the quality of the spatial representation can also be increased under certain circumstances by the inventive method.

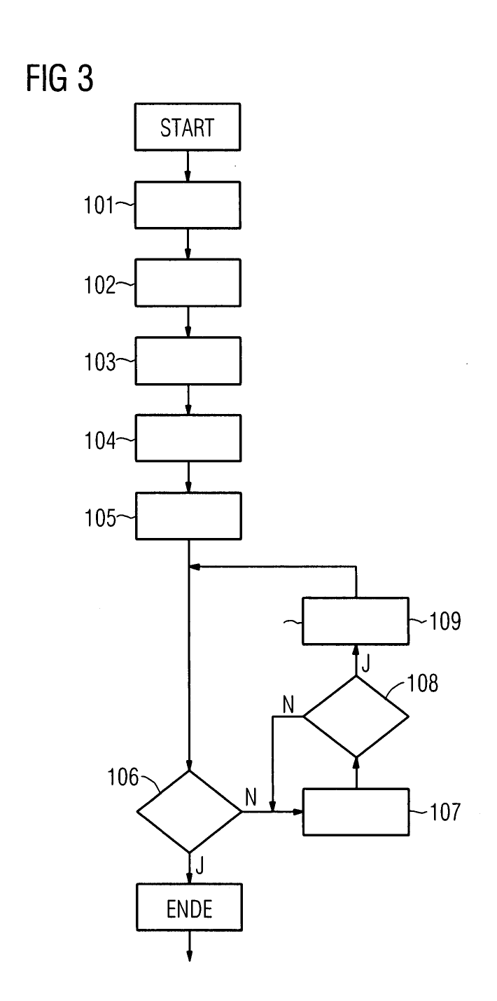

The flowchart in FIG. 3 shows an exemplary execution sequence for the inventive method and is explained in conjunction with the x-ray device 10 shown in FIG. 1, the reference numbers of device components referring to FIG. 1.

In order to avoid damage to the interfering object S, which is designed for example as an indicator light, and to prevent impairment of the results of the examination, the spatial representation of the area under examination of the object under examination U, planning of the recording sequence and/or the examination sequence takes place in a first method step 101.

For this purpose distance sensors (not shown) are advantageously provided in FIG. 1, which record the distance from potential interfering objects, such as for example interfering object S, to the recording device 20, and which feed the relative location and position with respect to movable device components, such as the recording device 20, to the control device 50. The result of the distance recording is presented graphically on the input/output unit 60 so that the relative position of the recording device 20 to the object under examination U, to the patient positioning device 70 and to a potential interfering object S is recorded. Medical personnel can subsequently graphically mark a spatial region in which the recording device 20 can safely move, as well as a spatial region that will not be irradiated with x-rays, for example with regard to the metal struts 71 shown in FIG. 1. In a next method step 102 the marked spatial region is fed to the control de-vice 50, which controls the drive device 24 such that the recording device 20 is only movable within the marked spatial region. Inadvertent collisions between the recording device 20 and the interfering object S can thus be eliminated, for example.

In a next method step 103 a simulation of the examination sequence is performed, which is generated for example by over-laying the rotary motions around the orbital axis O and the angulation axis A. In the event of a collision or an increased risk of collision occurring during the simulation, the planning of the examination sequence can be corrected. If the simulation runs successfully, the examination sequence is initiated in a next method step 104 and the recording device 20 begins recording a first projection data set in accordance with a method step 105 taking account of the circumstances and/or general conditions of the examination. A different examination model can then be initiated by the recording device 20 according to the prevailing conditions. For example an orbital rotary motion can be performed at a specified constant angular position taking account of the general conditions. If no further different projection data sets are to be recorded by means of rotation around the orbital axis O, in addition to the previously recorded projection data sets in which the angular position was held constant, a next adjacent angular position is initiated, through which the position of the axis of rotation is changed. Once again taking account of the prevailing circumstances, projection data sets are recorded in this angular position by means of rotary motion around the orbital axis O. The change in the axis of rotation according to method step 106 is advantageously continued incrementally or continuously while taking account of the prevailing circumstances, until a number of projection data sets is recorded that allows a reconstruction of the area under examination.

The flowchart shown in FIG. 3 does not use the examination model just described, but instead uses the examination model presented below. The recording device 20 simultaneously performs a rotary motion around the angulation axis A and around the orbital axis O while taking account of the prevailing circumstances. The axis of rotation of the recording device 20 thus continuously changes its spatial position. Before each additional controlled change in the position of the axis of rotation, a check is performed in a method step 106 to determine whether further projection data sets are necessary in order to obtain the spatial representation. If the recording of further projection data sets is necessary then the position of the axis of rotation, and thus of the projection geometry for the x-ray emitter 21 and the x-ray detector 22, are changed in a next method step 107. A check is subsequently performed in a next method step 108 to determine whether the projection geometry of the recording device 20 now captured has already been recorded by previously recorded projection data sets. If the present projection geometry matches the projection geometry of a previous projection data set recorded during this examination sequence, the projection geometry is changed further until it differs from a previous projection geometry. Alternatively a manipulation of the recording device can also already be determined on the basis of the present planning so that a query as to the identity of the projection geometry is not necessary. If the projection geometry of previously obtained projection geometries of the x-ray emitter 21 and x-ray detector 22 is different, the associated projection data set is re-corded in a method step 109 and fed to the control device 50. A query is then performed again to determine whether the number of recorded projection data sets is sufficient for the de-sired reconstruction. Method steps 106 to 109 are repeated until the number of projection data sets is sufficient to achieve the spatial representation of the area under examination of the object under examination U to the desired plane of quality. The inventive method then terminates once a sufficient number of projection data sets for the reconstruction is present.

Although modifications and changes may be suggested by those skilled in the arts it is the intention of the inventors to embody within the patent warranted hereon all changes and modifications as reasonably and properly come within the scope of their contribution to the art.

We claim as our invention:

1. A method for recording radiological projection data sets of an examination subject, comprising the steps of:
irradiating an examination subject with an x-ray beam that penetrates the subject, and detecting x-rays in said x-ray beam attenuated by the subject while rotating said x-ray beam around an axis, having a spatial position, that is substantially perpendicular to a central ray of said x-ray beam, said plurality of two-dimensional projection data sets including two successively acquired two-dimensional projection data sets; and
changing the spatial position of said axis between acquisition of said two successively acquired two-dimensional projection data sets.

2. A method as claimed in claim 1 comprising acquiring a first of said two successively acquired two-dimensional projection data sets by rotating said x-ray beam around a first, substantially horizontal axis, with a first rotary motion, and acquiring a second of said two successively acquired two-dimensional projection data sets by rotating said x-ray beam around a second, substantially horizontal axis oriented at a right angle to said first axis, with a second rotary motion overlaid on said first rotary motion.

3. A method as claimed in claim 2 wherein said first axis is an angulation axis and said second axis is an orbital axis.

4. A method as claimed in claim 2 wherein said first axis is an orbital axis and said second axis is an angulation axis.

5. A method as claimed in claim 1 comprising automatically controlling said changing of said spatial position of said axis.

6. A method as claimed in claim 1 comprising manually changing said spatial position of said axis.

7. A method as claimed in claim 1 comprising changing said spatial position of said axis by motorized control.

8. A method as claimed in claim 1 comprising electronically planning said changing of said spatial position of said axis prior to changing said spatial position of said axis.

9. A method as claimed in claim 8 comprising restricting said changing of said spatial position of said axis only to a change of said spatial position of said axis identified during said planning.

10. A method as claimed in claim 1 comprising electronically simulating said changing of said spatial position of said axis prior to changing said spatial position of said axis.

11. A method as claimed in claim 1 comprising acquiring said plurality of two-dimensional projection data sets using a data acquisition device having a first device component and a second device component, and prior to or during changing said spatial position of said axis, measuring a distance between said first device component and said second device component and changing said spatial position of said axis dependent on said distance.

12. A method as claimed in claim 1 comprising acquiring said plurality of two-dimensional projection data sets with a data acquisition device having a device component located in an environment that includes an interfering object, and comprising, prior to or during changing said spatial position of said axis, measuring a distance between said device component and said interfering object and changing said spatial position of said axis dependent on said distance.
